(19) United States
(12) Patent Application Publication
(10) Pub. No.: US 2005/0118206 A1
(43) Pub. Date: Jun. 2, 2005

(54) Surfactant-based gel as an injectable, sustained drug delivery vehicle

(76) Inventors: Andrew Sheung-King Luk, Castro Valley, CA (US); Stan Lam, Dublin, CA (US); Yuanpeng Zhang, Sunnyvale, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/985,228

(22) Filed: Nov. 10, 2004

Related U.S. Application Data
(60) Provisional application No. 60/519,989, filed on Nov. 14, 2003.

(51) Int. Cl. A61K 38/00; A61K 38/16; A61K 48/00; A61K 31/715

(52) U.S. Cl. 424/400; 514/2; 514/8; 514/54; 514/171; 514/44

(57) ABSTRACT

The present invention provides methods and compositions for the sustained delivery of beneficial agents. In certain embodiments, the invention provides compositions comprising a surfactant, a solvent, and a beneficial agent, wherein upon exposure to a hydrophilic environment, the surfactant and solvent form a viscous gel and the beneficial agent is dispersed or dissolved in the gel. In other embodiments, the invention provides compositions comprising a surfactant, a solvent, a hydrophilic media, and a beneficial agent, wherein the surfactant, solvent, and hydrophilic media form a viscous gel and the beneficial agent is dispersed or dissolved in the gel.

### CROSS REFERENCE TO RELATED APPLICATIONS

This application claims the benefit of U.S. Provisional Application No. 60/519,989, filed Nov. 14, 2003, the entirety of which is incorporated by reference herein.

### FIELD OF THE INVENTION

The present invention relates to compositions and methods for the sustained delivery of a beneficial agent. In certain embodiments, the invention relates to compositions comprising a surfactant, solvent, and beneficial agent wherein the composition forms a viscous gel upon exposure to a hydrophilic environment such as water or bodily fluids or tissues.

### BACKGROUND OF THE INVENTION

Implantable or injectable polymeric drug delivery vehicles have many drawbacks and have proven to be less than ideal means for the sustained delivery of beneficial agents. Implantable drug delivery systems that utilize thermoplastic or thermosetting biodegradable polymers have to be formed outside the body by incorporating the drug into the polymer and shaping the mixture into a form such as a cylinder, disc, or fiber. The implant must then be inserted into the body through an incision.

One means for avoiding the incision required for implantable drug delivery systems is the injection of small particles, microspheres, or microcapsules that contain an active agent that can be released into the body. Although these materials can be injected into the body with a syringe, due to their particulate nature, they do not form a continuous film or solid implant with the structural integrity needed for certain prostheses. In addition, given the large surface area of the microparticles, there is often a large initial drug release upon injection. Furthermore, protein and DNA-based drugs are often degraded during the encapsulation process due to the harsh solvents and temperature extremes used. The injection of microparticles also involves a two-step process where the microparticles must be reconstituted at the time of administration.

Polymer compositions utilized in injectable implants often use solvents/plasticizers that are very or relatively soluble in aqueous body fluids to promote rapid solidification of the polymer at the implant site and to promote diffusion of the drug from the implant Fast release of those solvents often results in an initial, rapid release of the beneficial agent from the polymer composition, particularly when the beneficial agent is soluble in the solvent and the solvent rapidly disperses into body fluids. The burst often results in a substantial portion of the beneficial agent, if not all, being released in a very short time, such as hours or one or two days.

The “burst” effect associated with microparticles and gels can be unacceptable, particularly in those circumstances where sustained delivery is desired, i.e., delivery of a beneficial agent over a period of a week or a month or more, or where there is a narrow therapeutic window and release of excess beneficial agent can result in adverse consequences to the subject being treated, or where it is necessary to mimic the naturally-occurring daily profile of beneficial agents, such as hormones and the like, in the body of patients being treated. In this respect, it is typical for conventional solvent-based compositions and microparticles to have a drug burst wherein 30% to 75% of the drug contained in the composition is released within one day of the initial injection.

Furthermore, rapid uptake of bodily fluids can result in polymer precipitation such that a hardened implant or one with a hardened skin is produced, resulting in the inner pores and much of the interior of the polymer being restricted from contact with body fluids. Although the drug is slowly diffused from such polymeric depots over time, the slow access of bodily fluids to the interior of the depot results in a significantly longer time for achieving complete biodegradation of the implanted polymers, which is undesirable for life-long chronic therapy.

A need thus exists in the art for compositions and methods that allow for the sustained delivery of beneficial agents that overcome the problems encountered in the art with respect to implantable or injectable polymeric drug delivery vehicles.

### SUMMARY OF THE INVENTION

The present invention relates to non-polymeric, easily-injectable, biocompatible and biodegradable compositions that act as sustained drug delivery vehicles in which initial drug burst is minimized. In certain embodiments, the present invention relates to injectable compositions for the sustained delivery of beneficial agents comprising a surfactant, a solvent, and a beneficial agent, wherein upon exposure to a hydrophilic environment, the surfactant and solvent form a viscous gel in which the beneficial agent is dispersed or dissolved. Other embodiments of the invention relate to methods for delivering a beneficial agent to a patient over a sustained period of time comprising administering an injectable composition to the patient that comprises a surfactant, a solvent, and a beneficial agent, wherein upon exposure to aqueous bodily fluids, the surfactant and solvent form a viscous gel in which beneficial agent is dispersed or dissolved.

In certain other embodiments, the invention relates to compositions for the sustained delivery of beneficial agents comprising a surfactant, a solvent, a hydrophilic media, and a beneficial agent, wherein the surfactant, solvent, and hydrophilic media form a viscous gel and the beneficial agent is dispersed or dissolved in the gel. Other embodiments of the invention relate to methods for delivering a beneficial agent to a patient over a sustained period of time comprising administering a composition to the patient that comprises a surfactant, a solvent, a hydrophilic media, and a beneficial agent, wherein the surfactant, solvent, and hydrophilic media form a viscous gel and the beneficial agent is dispersed or dissolved in the gel.

### BRIEF DESCRIPTION OF THE DRAWINGS

FIG. 1 depicts the in vitro release profiles obtained for several lysozyme formulations containing various solvents and surfactants.

### DETAILED DESCRIPTION OF ILLUSTRATIVE EMBODIMENTS

The term “beneficial agent,” as used herein, refers to any agent that effects a desired beneficial, often pharmacological, effect upon administration to a human or an animal, whether alone or in combination with other pharmaceutical excipients or inert ingredients.

The term “injectable,” as used herein, refers to compositions that are suitable for injection into skin or other tissues. Injectable compositions, for example, can be dispensed from syringes under normal conditions under normal pressure or from an autoinjector under elevated pressure.

The terms “sustained delivery,” “delivering over a sustained period of time,” and all variations thereof, as used herein, refer to the release over an extended period of time of a beneficial agent from a viscous gel according to the invention, which will generally occur over a period of one week or longer, and preferably over 30 days or longer. In certain embodiments of the invention, the sustained delivery of a beneficial agent occurs after administration of a composition according to the invention to a patient.

The phrase “dispersed or dissolved,” as used herein, refers to all means of establishing the presence of a beneficial agent in a composition according to the invention and includes dissolution, dispersion, suspension, and the like.

The term “patient,” as used herein, refers to an animal, mammal, or human being.

The terms “administering,” “administer,” and all variations thereof, refer to and include any means by which a composition of the invention is introduced into a patient. When administration is for the purpose of treatment, administration may be for either prophylactic or therapeutic purposes. When provided prophylactically, the composition is provided in advance of any symptom. When provided therapeutically, the composition is provided at (or shortly after) the onset of a symptom.

The term “viscous gel,” as used herein, refers to a composition of the invention having a viscosity of from about 100 to about 200,000 poise, preferably from about 500 to about 50,000 poise.

The term “hydrophilic media,” as used therein, refers to aqueous media, such as, for example, water, saline solution, a solution of one or more buffering agents, or bodily fluids or tissues. The term “hydrophilic environment,” as used therein, refers to an aqueous environment, such as, for example, that of bodily fluids or tissues of humans and animals.

In certain embodiments, the present invention relates to non-polymeric, easily-injectable, biocompatible and biodegradable compositions comprising one or more surfactants and at least one solvent that act as sustained drug delivery vehicles in which initial drug burst is minimized. In certain other embodiments, the invention relates to methods for the delivery of a beneficial agent to a patient over a sustained period of time comprising administering to the patient a composition comprising a solvent, a surfactant, and a beneficial agent.

Surfactants, which include a wide range of biological molecules such as lipids, are amphiphilic molecules that have a hydrophilic part and a hydrophobic part. Most lipids, including phospholipids, are only slightly surface-active, i.e., they have extremely low solubility in water and have a very low critical micelle concenrtation (CMC). PEGylated lipids are lipids that have been derivatized by covalent attachment to polyethylene glycol (PEG) of molecular weight ranging from about 1000 to about 50,000. PEGylated lipids and PEGylated phospholipids are amphiphilic, highly surface-active, and have a much higher CMC and solubility than traditional lipids. At high concentrations in the presence of solvent and water, PEGylated lipids and phospholipids form a variety of robust gel phases.

When dissolved in water or in a hydrophobic solvent, surfactants, including PEGylated phospholipids, self-aggregate to form a variety of microstructures such as micelles, vesicles, lamella, disks etc. to minimize the free energy of mixing. Depending upon the hydrophilic-hydrophobic balance (HLB) of the surfactant(s), the surfactant concentration, the nature of the aqueous medium, including its salt concentration, the nature of the solvent, and temperature, these ternary systems exhibit complex phase behavior. At low surfactant concentration, a rod-like micellar phase can lead to a viscoelastic solution. At high surfactant concentrations, the cubic phase, concentrated micellar phase, hexagonal phase, and some bicontinuous phases can all lead to the formation of gel-like structured phases.

Surfactants can form gel-like liquid crystalline phases in a ternary system that includes the surfactant, a hydrophobic solvent, and a hydrophilic media. Such gel phases can span a large portion of the phase diagram, i.e., a large compositional range, at various temperatures. In certain embodiments of the invention, a gel phase can be achieved by either mixing a surfactant, a hydrophobic solvent, and a hydrophobic media, or by premixing a hydrophobic solvent and a surfactant, and then subjecting the mixture to a hydrophilic environment by, for example, subcutaneous or intramuscular administration of the mixture, resulting in formation of a gel in situ. Both approaches can be used as a depot delivery platform for therapeutics for parenteral use and the former can be applied as a topical drug delivery platform.

In certain aspects, the present invention is directed to methods of systemically or locally administering a beneficial agent to a patient by administering a composition, formed as a viscous gel from a surfactant and a solvent, in which a beneficial agent is substantially dissolved or dispersed throughout. The beneficial agent is released to the patient over a prolonged period of time, thus providing for delivery of the beneficial agent with a controlled burst of beneficial agent and sustained release thereafter.

The present invention further relates to compositions for the sustained delivery of beneficial agents in which a gel-like liquid crystalline phase is formed by one or more surfactants and a solvent in the presence of a hydrophilic media or in a hydrophilic environment, and the beneficial agent is substantially dissolved or dispersed in the gel. In preferred embodiments, the compositions of the invention exist in a liquid crystalline phase having a viscosity high enough for gel formation, yet low enough for processability and injectability under normal storage and delivery temperatures via either syringes or autoinjectors.

In certain aspects, the invention relates to compositions for the sustained delivery of a beneficial agent that comprise a surfactant, solvent, and beneficial agent. Upon exposure of the composition to a hydrophilic environment, a viscous gel is formed in which the beneficial agent is dissolved or dispersed. Other embodiments of the invention relate to methods for delivering a beneficial agent to a patient over a sustained period of time that comprise administering to the patient a composition comprising a surfactant, solvent, and beneficial agent. Upon diffusion of aqueous bodily fluids into the composition, a viscous gel is formed in which the beneficial agent is dissolved or dispersed. The beneficial agent is then released from the viscous gel over a sustained period of time.

In other aspects, the invention relates to compositions for the sustained delivery of a beneficial agent that comprise a surfactant, solvent, hydrophilic media, and beneficial agent, wherein the surfactant, solvent, and hydrophilic media form a viscous gel in which the beneficial agent is dissolved or dispersed. Other embodiments of the invention relate to methods for delivering a beneficial agent to a patient over a sustained period of time that comprise administering to the patient a composition comprising a surfactant, solvent, hydrophilic media, and beneficial agent. Such compositions form a viscous gel and can be administered by injection or topically.

Suitable surfactants for use in the methods and compositions of the invention include, for example, ionic surfactants (possessing at least one ionized moiety) and nonionic surfactants (having no ionized groups). Ionic surfactants include, without limitation, anionic surfactants, such as fatty acids and salts of fatty acids (e.g., sodium lauryl sulfate); sterol acids and salts thereof (e.g., cholate and deoxycholate); cationic surfactants, such as alkyl tri-methyl and ethyl ammonium bromides (e.g., cetyl triethyl ammonium bromide (CTAB) and C₁₆TAB); amphoteric surfactants, such as lysolipids (e.g., lysophosphatidylcholine or phosphatidylethanolamine), and CHAPS; and Zwittergents, such as Zwittergent® 3-14.

Nonionic surfactants suitable for use in the methods and compositions of the invention include, without limitation, fatty alcohols, that is, alcohols having the structural formula CH₃(CH₂)ₙC(H)OH (e.g, where n is at least 6), such as lauryl, cetyl and stearyl alcohols; fatty sugars, such as octyl glucoside and digitonin; Lubrols, such as Lubrol® PX; Tritons, such as TRITON® X-100; Nonidents, such as Nonident P-40; sorbitan fatty acid esters (such as those sold under the tradename SPAN®), polyoxyethylene sorbitan fatty acid esters (such as those sold under the tradename TWEEN®), polyoxyethylene fatty acid esters (such as those sold under the tradename MYRJ®), polyoxyethylene steroidal esters, polyoxypropylene sorbitan fatty acid esters, polyoxypropylene fatty acid esters, polyoxypropylene steroidal esters, polyoxyethylene ethers (such as those sold under the tradename BRIJ®), polyglycol ethers (such as those sold under the tradename TERGITOL®), and the like. Preferred nonionic surfactants are polyglycol ethers, polyoxyethylene sorbitan trioleate, sorbitan monopalmitate, polysorbate 80, polyoxyethylene 4-lauryl ether, propylene glycol, and mixtures thereof.

Anionic surfactants suitable for use in the methods and compositions of the invention include, without limitation, long-chain alkyl sulfonates, carboxylates, and sulfates, as well as alkyl aryl sulfonates, and the like. Preferred anionic surfactants are sodium dodecyl sulfate, dialkyl sodium sulfosuccinate (e.g., sodium bis-(2-ethylhexyl)-sulfosuccinate), sodium 7-ethyl-2-methyl-4-dodecyl sulfate and sodium dodecylbenzene sulfonate.

Cationic surfactants suitable for use in the methods and compositions of the invention include, without limitation, long-chain amine salts or quaternary ammonium salts, e.g., decyltrimethylammonium bromide, dodecyltrimethylammonium bromide, tetradecyltrimethylammonium bromide, tetradecyltrimethylammonium chloride, and the like.

Amphoteric surfactants suitable for use in the methods and compositions of the invention include, without limitation, compounds that include a carboxylate or phosphate group as the anion and an amino or quaternary ammonium moiety as the cation. These include, for example, various polypeptides, proteins, alkyl betaines, and natural phospholipids such as lysolecithins and lysocephalins.

Preferred surfactants include, but are not limited to, phospholipids, PEGylated phospholipids, PEGylated lipids, polyoxyethylene-polyoxypropylene copolymers, ethoxylated sorbitan esters, sorbitan esters, ethoxylated ethers, ethoxylated castor oils, vitamin E-TPGS (D-α-tocopheryl PEG 1000 succinate), sphingolipids, glycolipids, lysophospholipids, fatty acids, bile salts, ethoxylated glycerides, ethoxylated fatty alcohols, and mixtures thereof.

PEGylated lipids suitable for use in the methods and compositions of the invention include, for example, PEG-DSPE (polyethyleneglycol conjugated to distearoylphosphatidylethanolamine), mPEG-DS (methylether-polyethyleneglycol conjugated to distearoyl), and PEG-ceramides.

The lipids and phospholipids that serve as surfactants in the compositions and methods of certain embodiments of the invention can be conjugated to polymers other than PEG such as, for example, polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, polyethyleneoxide-polypropylene oxide copolymers, copolymers of the above-recited polymers, and mixtures thereof. Lipids and phospholipids conjugated to any of the foregoing polymers can thus serve as a surfactant in the methods and compositions of certain embodiments of the invention.

In certain embodiments of the invention, the compositions comprise from about 5% to about 80% by total weight of the surfactant. In certain more preferred embodiments, the compositions of the invention comprise from about 10% to about 70% by total weight of the surfactant. In even more preferred embodiments, the compositions of the invention comprise from about 15% to about 60% by total weight of the surfactant.

Suitable solvents for use in the methods and compositions of the invention include hydrophilic, aprotic, and hydrophobic solvents. Preferred solvents include, but are not limited to, ethyl oleate, benzyl benzoate, ethyl benzoate, lauryl lactate, benzyl alcohol, lauryl alcohol, glycofurol, ethanol, tocopherol, polyethylene glycol, triacetin, triglycerides, alkyltriglycerides, diglycerides, sesame oil, peanut oil, castor oil, olive oil, cottonseed oil, perfluorocarbon, N-methyl-pyrrolidone, DMSO, glycerol, oleic acid, glycofurol, lauryl lactate, perfluorocarbon, propylene carbonate, and mixtures thereof.

In certain embodiments of the invention, the compositions comprise from about 20% to about 95% by total weight of the solvent. In certain more preferred embodiments, the compositions of the invention comprise from about 30% to about 90% by total weight of the solvent. In even more preferred embodiments, the compositions of the invention comprise from about 40% to about 55% by total weight of the solvent.

Suitable beneficial agents for use in the methods and compositions of the invention include any physiologically or pharmacologically active substance or substances, optionally in combination with pharmaceutically acceptable carriers and additional ingredients such as antioxidants, stabilizing agents, permeation enhancers, etc. that do not substantially adversely affect the advantageous results that can be attained with the compositions and methods of the present invention. The beneficial agent may include drug agents, medicaments, vitamins, nutrients, or the like. Included among the types of agents that meet this description are lower molecular weight compounds, proteins, peptides, genetic material, nutrients, vitamins, food supplements, sex sterilants, imaging agents, fertility inhibitors and fertility promoters. Preferred beneficial agents include, for example, proteins, peptides, enzymes, hormones, polynucleotides, nucleoproteins, polysaccharides, glycoproteins, lipoproteins, polypeptides, small molecules including, but not limited to, steroids, analgesics, local anesthetics, antibiotic agents, anti-inflammatory corticosteroids, anti-microbial agents, contrast agents, such as, for example, Gd-DTPA (Gadolinium (III) diethyltriaminepentaacetic acid), gadodiamide, gadoteridol, Gd-DTPA-labeled albumin, Gd-DTPA-labeled dextran, and chromium-labeled red blood cells, ocular drugs, and chemotherapeutic agents.

Beneficial agents that can be used in the methods and compositions of the present invention include drugs that act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system.

Examples of beneficial agents that can be used in the compositions and methods of the present invention include, but are not limited to, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17-α-hydroxyprogesterone acetate, 19-nor-progesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, eryhromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth hormones such as human growth hormone and its derivatives such as methione-human growth hormone and des-phenylalanine human growth hormone, bovine growth hormone and porcine growth hormone, fertility inhibitors such as the prostaglandins, fertility promoters, growth factors such as insulin-like growth factor, coagulation factors, human pancreas hormone releasing factor, analogs and derivatives of these compounds, and pharmaceutically acceptable salts of these compounds, or their analogs or derivatives.

In certain embodiments, the present invention also finds application with chemotherapeutic agents for the local application of such agents to avoid or minimize systemic side effects. In some embodiments, gels of the present invention containing chemotherapeutic agents can be injected directly into the tumor tissue for sustained delivery of the chemotherapeutic agent over time. In some embodiments, particularly after resection of the tumor, the gel can be implanted directly into the resulting cavity or can be applied to the remaining tissue as a coating. In embodiments in which the gel is implanted after surgery, it is possible to utilize gels having higher viscosities since they do not have to pass through a small diameter needle. Representative chemotherapeutic agents that can used in the methods and compositions of certain embodiments of the present invention include, for example, carboplatin, cisplatin, paclitaxel, BCNU, vincristine, camptothecin, etopside, cytokines, ribozymes, interferons, oligonucleotides and oligonucleotide sequences that inhibit translation or transcription of tumor genes, functional derivatives of the foregoing, and generally known chemotherapeutic agents such as those described, for example, in U.S. Pat. No. 5,651,986, incorporated herein by reference in its entirety. In certain embodiments of the invention, the compositions and methods have particular utility in the sustained delivery of water soluble chemotherapeutic agents, such as, for example cisplatin and carboplatin and the water soluble derivatives of paclitaxel. The characteristics of certain embodiments of the invention that minimize the burst effect are particularly advantageous in the administration of water soluble beneficial agents of all kinds, but particularly those compounds that are clinically useful and effective but may have adverse side effects.

To the extent not mentioned above, the beneficial agents described in U.S. Pat. No. 5,242,910, incorporated herein by reference in its entirety, can also be used in the compositions and methods of certain embodiments of the invention.

Notably, materials, such as proteins, as exemplified by the enzyme lysozyme, cDNA, and DNA incorporated into vectors both viral and nonviral, which are difficult to microencapsulate or process into microspheres, can be incorporated into the compositions of the present invention without the level of degradation caused by exposure to high temperatures and denaturing solvents often present in other processing techniques.

In certain embodiments of the invention, the beneficial agent is formulated into particles by, for example, dry milling, wet milling, micronization, lyophilization, spay drying, spray-freeze drying, homogenization, or super critical fluid micronization. In some embodiments of the invention, the particles are coated to provide further control of the release of the beneficial agent. In certain embodiments of the invention, the particles of the beneficial agent comprise stabilizers such as, for example, sucrose, tehalose, mannitol, and glycine; buffers such as, for example, phosphate, histidine, and succinate; or antioxidants such as, for example, vitamin E or methionine. In some embodiments of the invention, the particles of the beneficial agent comprise one or more stabilizers, buffets, antioxidants, or combinations thereof. In certain embodiments of the invention, the particles of the beneficial agent can be complexed with another molecule such as a Zinc salt, or one or more polymers, for stabilization.

In preferred embodiments of the invention, the beneficial agent is incorporated into the viscous gel formed from the surfactant, solvent, and hydrophilic media in the form of particles typically having an average particle size of from about 0.1 to about 200 microns, preferably from about 1 to about 125 microns, and often from about 2 to about 100 microns.

To form a suspension or dispersion of particles of the beneficial agent in the viscous gel formed from the surfactant, solvent, and hydrophilic media, any conventional low shear device can be used such as a double planetary mixer. In this manner, efficient distribution of the beneficial agent can be achieved substantially without degrading the beneficial agent.

In preferred embodiments, the beneficial agent is typically dissolved or dispersed in the compositions of the invention in an amount of from about 1% to about 50% by total weight, preferably in an amount of from about 5% to about 40% by total weight, and often from about 10% to about 30% by total weight. Depending upon the amount of beneficial agent present in the composition, different release profiles and burst indices can be obtained.

In certain embodiments of the invention, the release rate and loading of the beneficial agent are adjusted to provide for therapeutically-effective delivery of the beneficial agent over the intended sustained delivery period. Preferably, the beneficial agent is present in the gel at concentrations that are above the saturation concentration of the beneficial agent in water. While the release rate of the beneficial agent depends upon the particular circumstances, such as the particular beneficial agent to be administered, release rates on the order of from about 0.1 micrograms per day to about 100 milligrams per day, preferably from about 10 micrograms per day to about 10 milligrams per day, for periods of from about 7 to about 90 days can be obtained. Greater amounts of the beneficial agent can be delivered if delivery is to occur over shorter periods of time. Generally, higher release rates are possible if a greater burst can be tolerated. Further, the dose of the beneficial agent can be controlled by adjusting the volume of the composition that is implanted or injected.

In certain embodiments of the invention, the hydrophilic media includes, but is not limited to, water, saline solution, a solution of one or more buffering agents, bodily fluids, or bodily tissues. Hydrophilic environments include, but are not limited to, aqueous environments, such as, for example, that of bodily fluids or tissues of humans or animals.

In embodiments of the invention in which the compositions comprise a surfactant, solvent, hydrophilic media, and beneficial agent, the compositions comprise from about 0.1% to about 10% by total weight of the hydrophilic media. In certain more preferred embodiments, such compositions comprise from about 0.1% to about 2% by total weight of the hydrophilic media. In even more preferred embodiments, such compositions comprise from about 0.1% to about 0.5% by total weight of the hydrophilic media.

In certain embodiments, other components may be present in the compositions of the invention, to the extent that such additional components are desired or provide useful properties to the compositions, such as, for example, polyethylene glycol, hydroscopic agents, stabilizing agents, buffering agents, pore forming agents, viscosity-building agents, and others. When the compositions include a peptide or a protein that is soluble in or unstable in an aqueous environment, it may be highly desirable to include a solubility modulator, that may, for example, be a stabilizing agent, in the compositions. Various solubility modulating agents are described, for example, in U.S. Pat. Nos. 5,654,010 and 5,656,297 which are incorporated herein by reference in their entireties. In the case of hGH, for example, it is preferable to include an amount of a salt of a divalent metal, preferably zinc. Examples of such solubility modulators and stabilizing agents, which may form complexes with the beneficial agent or associate with it to provide the stabilizing or modulated release effect, include metal cations, preferably divalent, present in the composition as magnesium carbonate, zinc carbonate, calcium carbonate, magnesium acetate, magnesium sulfate, zinc acetate, zinc sulfate, zinc chloride, magnesium chloride, magnesium oxide, magnesium hydroxide, other antacids, and the like. The amount of such agents used will depend upon the nature of the complex formed, if any, or the nature of the association between the beneficial agent and the agent. In certain embodiments of the invention, molar ratios of solubility modulator or stabilizing agent to beneficial agent of about 100:1 to 1:1, preferably 10:1 to 1:1, typically can be utilized.

In certain embodiments of the invention, the compositions can comprise agents that stabilize macromolecules, such as, for example, lyoprotectants, including, but not limited to, trehalose, sucrose, and glycine. In some embodiments, the compositions of the invention can comprise buffering agents such as, for example, phosphates, succinates, histidines, and acetate. In further embodiments, the compositions of the invention can include an additional surfactant, such as Tween 20.

In certain embodiments, a viscosity-building agent can be dispersed or dissolved in the compositions of the invention to increase the viscosity of the compositions, resulting in their stabilization. Viscosity-building agents include, but are not limited to, polymers such as polyvinylpyrrolidone, methyl cellulose, ethyl cellulose, hydroxyl ethyl starch, poly-lactide-glycolic acid, poly caprolactone-LA-GA copolymers. In certain embodiments of the invention, the compositions comprise from about 0.1% to about 5% by total weight of the viscosity-building agent.

In certain embodiments of the invention, the compositions are highly viscous prior to implantation or injection. The viscosity of the compositions can be lowered by dispersing or dissolving an additional surfactant in the compositions, reducing the viscosity enough to permit passage of the compositions through a needle. Also, pore formers and solubility modulators of the beneficial agent can be added to the compositions to provide desired release profiles, along with typical pharmaceutical excipients and other additives that do not change the beneficial aspects of the compositions of certain embodiments of the present invention.

Since, in certain embodiments, the compositions of the present invention are preferably formed as viscous gels, the means of administration of the compositions is not limited to injection, although that mode of delivery may often be preferred. In certain embodiments of the invention, the compositions can be administered by surgical implantation. In other embodiments, the compositions of the invention can be applied topically to the skin or other tissues. Where the compositions are administered as a leave-behind product, they can be formed to fit into a body cavity existing after completion of surgery or they can be applied as a flowable gel by brushing or palleting the gel onto residual tissue or bone. Such applications can permit loading of beneficial agent in the gel above concentrations typically present with injectable compositions.

Certain embodiments of the invention relate to compositions that do not include a beneficial agent, which can be used for wound healing, bone repair, and other structural support purposes.

In certain embodiments, certain preferred compositions of the invention comprise 5% to 80% by total weight of a surfactant, 20% to 95% by total weight of a solvent, and 1% to 50% by total weight of a beneficial agent. In other embodiments, preferred compositions of the invention comprise 5% to 80% by total weight of a surfactant, 20% to 95% by total weight of a solvent, 0.1% to 10% by total weight of hydrophilic media and 1% to 50% by total weight of a beneficial agent

The following examples are illustrative of certain embodiments of the invention and should not be considered to limit the scope of the invention.

### EXAMPLE 1

### Preparation of a Composition for the Sustained Delivery of Lysozyme

Sample Preparation

Four test samples and two control samples were prepared as follows. For each sample, a surfactant and a solvent in the weight ratio described in the table below were mixed in a 20 ml scintillation vial. The total weight of the surfactant and solvent in each sample was approximately 5 grams. The samples were then mixed using a Keyence Hybrid mixer for 10 minutes. Lysozyme (17 970 u/mgDW from Worthington) was then added to each vial in a dry box until the viscosity of the mixture increased to a level that was sufficient to allow the samples to be loaded into release cells, resulting in the addition of approximately 1 gram of lysozyme to each sample. The vials were tightly sealed and placed in a Keyence mixer for one minute. The vials were then stirred using an overhead mixer until a homogeneous mixture was obtained. A clear gel phase was obtained for the hydrogel formulation (Pluronic F127/water/butanol).

| | Formulation | Lysozyme | Wt % |
|---|---|---|---|
| Sample | (mg) | (mg) | lysozyme |
| Pluronic F127/water/butanol (18/72/10) | 4.1682 | 0.8668 | 20.80 |
| Reverse pluronic 31R1/benzyl benzoate (80/20) | 5.1432 | 0.8557 | 16.64 |
| Pluronic L62/benzyl benzoate (80/20) | 3.8020 | 0.8205 | 21.58 |
| Polysorbate 80/benzyl benzoate (80/20) | 5.5760 | 1.3730 | 24.62 |
| Polyvinylpyrrolidone/benzyl benzoate (50/50): fast | 4.1008 | 1.0001 | 24.39 |
| release control | | | |
| Poly(lactide-co-glycolide)/benzyl benzoate (25/75) with | 4.5522 | 1.0458 | 22.97 |
| a 2 wt % Pluronic F68 depot: controlled release | | | |
| control | | | |

In Vitro Release Testing

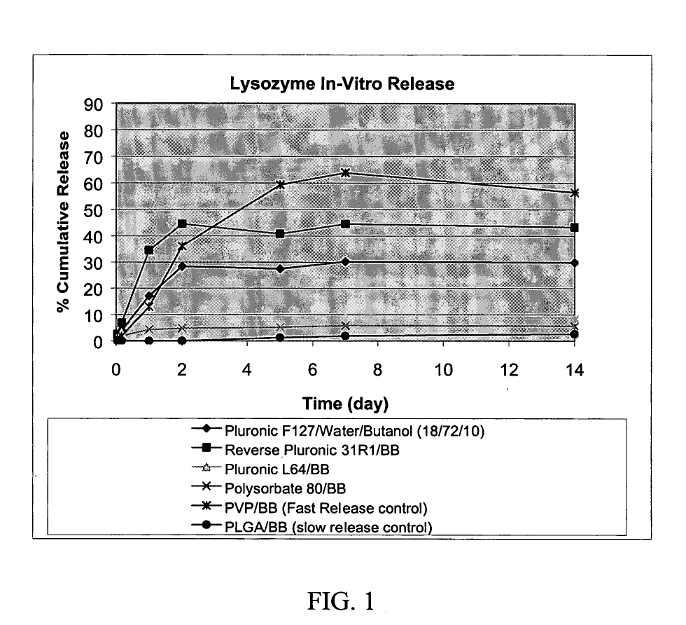

Samples were loaded into a 500 mg in vitro release cell. The temperature of the bath was maintained at 37° C. and the pH was maintained at 7.4. Samples were removed after one hour, four hours, one day, two days, three days, five days, seven days, ten days, and fourteen days and analyzed using HPLC to determine protein concentration. As shown in FIG. 1, at day fourteen the four test samples exhibited a slower release rate than that of the fast release control, and also exhibited a faster release rate than that of the controlled release control. At day fourteen the reverse pluronic 31R1/benzyl benzoate sample exhibited the fastest release rate of the test samples, the pluronic F127/water/butanol sample exhibited the second fastest release rate of the test samples, the pluronic L64/benzyl benzoate sample exhibited the third fastest release rate of the test samples, and the polysorbate 80/benzyl benzoate exhibited the slowest release rate of the test samples.

### EXAMPLE 2

### Preparation of a Composition for the Sustained Delivery of Human Growth Hormone

Particles of recombinant human growth hormone are prepared by lyophilization of recombinant human growth hormone, sucrose, glycine, and phosphate in the amounts indicated below, followed by particle reduction and sizing, and/or spray drying. A gel is then prepared by mixing polysorbate 20 and benzyl benzoate (in the amounts indicated below) in a double plenatary mixer until the polysorbate 20 is dissolved in the benzyl benzoate. Polyvinylpyrrilidone in the amount indicated below is then slowly added to the polysorbate 20/benzyl benzoate mixture form a viscous gel. The human growth hormone particles are then slowly dispersed into the gel to form a dosage form.

| Ingredients | Function | Composition ranges |
|---|---|---|
| Polysorbate 20 | Surfactant | 9.9%-69.9% |
| Benzyl Benzoate | Solvent | 9.9%-69.9% |
| Polyvinylpyrrilidone | Viscosity-building agent | 0.1%-1.0% |
| (PVP) | | |
| Human growth hormone | Therapeutic agent | 10% |
| Sucrose/Glycine | Stabilizer | 8% |
| Phosphate | Buffer | 2% |

### EXAMPLE 3

### Preparation of a Composition for the Sustained Delivery of Lysozyme

A surfactant/solvent mixture containing 80% by weight of Polysorbate 80 (Croda) and 20% by weight of benzyl benzoate (Charkit) having a total weight of 10 grams is prepared in a 20 ml scintillation vial by mixing the two components with either an overhead mixer or a spatula. Lysozyme particles are prepared by spray drying a lysozyme solution, resulting in lysozyme particles of approximately 5 microns, or by grinding and sieving lyophilized lysozyme cakes, resulting in lysozyme particles of approximately 38 to 125 microns. One to two grams of lysozyme particles are weighed and fully dispersed in the surfactant/solvent mixture using an overhead mixer or spatula, which increases the viscosity of the formulation. The formulation is loaded into an in vitro release cell, and the release profile is obtained.

### EXAMPLE 4

### Preparation of a Composition for the Sustained Delivery of Monoclonal Antibody

A surfactant/solvent mixture containing 60% by weight of Pluronic L64 (BASF) and 40% by weight of benzyl benzoate (Charkit) having a total weight of 40 grams is prepared in a small double-plenatary mixer. Ten grams of povidone 17 pF or polyvinylpyrrolidone (BASF) is added to the solvent/surfactant mixture to increase the viscosity of the formulation. Polyvinylpyrrolidone is allowed to dissolve in the vehicle. Monoclonal antibody particles having a particle size of approximately 5 to 10 microns are prepared by spray drying, are added to the viscous vehicle, and are dispersed by a double plenatary mixer under a vacuum to prevent the formation of air bubbles. The resultant formulation is transferred to a large HDPE syringe where it is filled into small 0.5-ml glass syringes.

### EXAMPLE 5

### Preparation of a Composition for the Sustained Delivery of Recombinant Human Growth Hormone

A surfactant/solvent mixture containing 50% by weight of Cremophor ELP (BASF) and 50% by weight of Castor oil (Croda) having a total weight of 500 grams is prepared in a large-scale double plenatary mixer. Solid recombinant human growth hormone (rhGH) particles are dispersed homogeneously into the surfactant/solvent mixture by continuous mixing under a vacuum. The formulation is transferred in a closed system to a filling cartridge, where individual syringes or vials are filled.

The entire disclosure of each patent, patent application, and publication cited or described in this document is hereby incorporated herein by reference.

1. An injectable composition for the sustained delivery of a beneficial agent comprising a surfactant, a solvent, and a beneficial agent, wherein upon exposure to a hydrophilic environment, the surfactant and solvent form a viscous gel and the beneficial agent is dispersed or dissolved in the gel.

2. The composition of claim 1 wherein the surfactant is anionic, cationic, zwitterionic, or nonionic.

3. The composition of claim 2 wherein the surfactant is a phospholipid, a PEGylated phospholipid, a polyoxyethylene-polyoxypropylene copolymer, an ethoxylated sorbitan ester, a sorbitan ester, an ethoxylated ether, an ethoxylated castor oil, D-α-tocopheryl PEG 1000 succinate, a sphingolipid, a glycolipid, a lysophospholipid, a fatty acid, a bile salt, an ethoxylated glyceride, an ethoxylated fatty alcohol, or mixtures thereof.

4. The composition of claim 1 comprising 5% to 80% by total weight of the surfactant.

5. The composition of claim 1 wherein the solvent is hydrophobic.

6. The composition of claim 5 wherein the hydrophobic solvent is ethyl oleate, benzyl benzoate, ethyl benzoate, lauryl lactate, benzyl alcohol, lauryl alcohol, glycofurol, ethanol, tocopherol, polyethylene glycol, triacetin, a triglyceride, an alkyltriglyceride, a diglyceride, sesame oil, peanut oil, castor oil, olive oil, cottonseed oil, perfluorocarbon, N-methyl-pyrrolidone, DMSO, glycerol, oleic acid, glycofurol, lauryl lactate, perfluorocarbon, propylene carbonate, or mixtures thereof.

7. The composition of claim 1 comprising 20% to 95% by total weight of the solvent.

8. The composition of claim 1 wherein the beneficial agent is a protein, peptide, enzyme, hormone, polynucleotide, nucleoprotein, polysaccharide, glycoprotein, lipoprotein, polypeptide, steroid, analgesic, local anesthetic, antibiotic agent, anti-inflammatory corticosteroid, anti-microbial agent, contrast, agent, ocular drug, or chemotherapeutic agent.

9. The composition of claim 1 comprising 1% to 50% by total weight of the beneficial agent.

10. The composition of claim 1 wherein the hydrophilic environment comprises water, saline solution, or bodily fluids or tissues.

11. The composition of claim 1 comprising 5% to 80% by total weight of the surfactant, 20% to 95% by total weight of the solvent, and 1% to 50% by total weight of the beneficial agent.

12. A composition for the sustained delivery of a beneficial agent comprising a surfactant, a solvent, a hydrophilic media, and a beneficial agent, wherein the surfactant, solvent, and hydrophilic media form a viscous gel and the beneficial agent is dispersed or dissolved in the gel.

13. The composition of claim 12 wherein the surfactant is anionic, cationic, zwitterionic, or nonionic.

14. The composition of claim 13 wherein the surfactant is a phospholipid, a PEGylated phospholipid, a polyoxyethylene-polyoxypropylene copolymer, an ethoxylated sorbitan ester, a sorbitan ester, an ethoxylated ether, an ethoxylated castor oil, vitamin E-TPGS, a sphingolipid, a glycolipid, a lysophospholipid, a fatty acid, a bile salt, an ethoxylated glyceride, an ethoxylated fatty alcohol, or mixtures thereof.

15. The composition of claim 12 comprising 5% to 80% by total weight of the surfactant.

16. The composition of claim 12 wherein the solvent is hydrophobic.

17. The composition of claim 16 wherein the hydrophobic solvent is ethyl oleate, benzyl benzoate, ethyl benzoate, lauryl lactate, benzyl alcohol, lauryl alcohol, glycofurol, ethanol, tocopherol, polyethylene glycol, triacetin, a triglyceride, an alkyltriglyceride, a diglyceride, sesame oil, peanut oil, castor oil, olive oil, cottonseed oil, perfluorocarbon, propylene carbonate, N-methyl-pyrrolidone, DMSO, glycerol, oleic acid, glycofurol, lauryl lactate, perfluorocarbon, or mixtures thereof.

18. The composition of claim 12 comprising 20% to 95% by total weight of the hydrophobic solvent.

19. The composition of claim 12 wherein the hydrophilic media is water, saline solution, bodily fluids, or bodily tissues.

20. The composition of claim 12 comprising 0.1% to 10% by total weight of the hydrophilic media.

21. The composition of claim 12 wherein the beneficial agent is a protein, peptide, enzyme, hormone, polynucleotide, nucleoprotein, polysaccharide, glycoprotein, lipoprotein, polypeptide, steroid, analgesic, local anesthetic, antibiotic agent, anti-inflammatory corticosteroid, anti-microbial agent, contrast agent, ocular drug, or chemotherapeutic agent.

22. The composition of claim 12 comprising 1% to 50% by total weight of the beneficial agent.

23. The composition of claim 12 comprising 5% to 80% by total weight of the surfactant, 20% to 95% by total weight of the solvent, 0.1% to 10% of total weight of the hydrophilic media, and 1% to 50% by total weight of the beneficial agent.

24. A method for delivering a beneficial agent to a patient over a sustained period of time comprising administering to the patient the composition of claim 1.

25. The method of claim 24 wherein the composition is delivered to the patient by injection.

26. A method for delivering a beneficial agent to a patient over a sustained period of time comprising administering to the patient the composition of claim 12.

27. The method of claim 26 wherein the composition is delivered to the patient by injection or topically.
